(19) United States
(12) Patent Application Publication
(10) Pub. No.: US 2005/0113302 A1
(43) Pub. Date: May 26, 2005

(54) A3 receptor-mediated cardioprotective proteins and therapeutic and diagnostic methods of use

(76) Inventors: John F. Thompson, Warwick, RI (US); David M. Potter, Mystic, CT (US); Delvin R. Knight JR., Ledyard, CT (US); Antonio Gualberto, East Greenwich, CT (US); Katherine T. Landschulz, East Lyme, CT (US); Herath Mudiyenselage Athula Chandrasiri Herath, Abingdon (GB); Christian Rohlff, Abingdon (GB)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/652,779

(22) Filed: Aug. 29, 2003

Related U.S. Application Data
(60) Provisional application No. 60/406,850, filed on Aug. 29, 2002.

(51) Int. Cl. A61K 38/10; A61K 38/08; C07K 7/06; C07K 7/08

(52) U.S. Cl. 514/14; 514/15; 514/16; 530/326; 530/327; 530/328; 530/329

(57) ABSTRACT

Proteins are identified that exhibit an altered expression in mammalian cardiac tissue in response to A₃ agonist infusion. These proteins are termed A₃ receptor-mediated cardioprotective proteins (A₃Ps) and protein isoforms (A₃PIs) and are useful as pharmaceuticals and as targets in screening methods for identifying an agent capable of modulating the expression of one or more A₃P and/or A₃PI.

### RELATED APPLICATIONS

This application claims the benefit of priority to U.S. Provisional Application Ser. No. 60/406,850, filed Aug. 29, 2002; the entirety of which is hereby incorporated by reference.

### FIELD OF THE INVENTION

This invention relates to use of proteins identified as exhibiting an altered expression and/or activity in response to activation of adenosine A₃ receptors or administration of A₃ agonists. The proteins identified are useful for clinical screening, diagnosis, prognosis, therapy, and prophylaxis. These proteins are also useful in screening assays for identification of therapeutic molecules capable of providing protection from cardiac and non-cardiac ischemic injury.

### BACKGROUND

Cardiovascular disease, including cardiac ischemic injury induced by inhibition of coronary blood flow and oxygen transport, is a leading cause of mortality and morbidity in patients. Protection against myocardial ischemia injury leading to myocardial infarction (dead heart tissue) remains a major goal for cardiology.

Ischemia, however, may be protective as well as injurious. Ischemic preconditioning is a phenomenon whereby a brief period of ischemia renders the myocardium resistant to myocardial injury, including infarction from a subsequent prolonged ischemic insult. The phenomenon of ischemic preconditioning, which occurs in brain and kidney tissue as well as the heart, has been reported to be mediated through the A₃ adenosine receptor. However, the mechanism for how reduction of cardiac and non-cardiac ischemic injury is achieved through activation of adenosine A₃ receptors is not fully known.

U.S. Pat. No. 5,573,772 (Downey et al.) describes an A₃ adenosine receptor as the mediator of ischemic preconditioning. U.S. Pat. No. 5,604,210 (Nagaoka et al.) describes compounds potentially useful for the prevention or treatment of brain edema and cerebral infarction. U.S. Pat. No. 5,688,774 (Jacobson et al.) describes A₃ receptor agonists, particularly adenine compounds having selected substituents at the 2, 6, and 9 positions. U.S. Pat. No. 5,773,423 (Jacobson et al.) describes A₃ receptor agonists such as N⁶-benzyladenosine-5′-uronamide, and related substituted compounds. Gallo-Rodriguez et al. (1994) J. Med. Chem. 37:636-646 describe the synthesis of adenosine analogues modified at the 5′ position as uronamides and/or as N⁶-benzyl derivatives having varying affinity for A₃ receptors. Jacobson et al. (1995) J. Med. Chem. 38:1174-1188 describes binding affinities of a variety of adenosine derivatives for rat A₁, A₂ₐ, and A₃ receptors. Jacobson et al. (1995) J. Med. Chem 38:1720-1735 describe 9-alkyladenine derivatives and ribose-modified N⁶-benzyl derivatives having affinity for the A₃ receptor with potential usefulness as leads for development of A₃ receptor antagonists. U.S. Pat. No. 5,817,760 (Jacobson et al.) describes recombinant human A₁, A₂ₐ, and A₃ receptors. WO 01/23399 describes compounds useful for preventing tissue damage and/or inducing tissue protection mediated through the A₃ adenosine receptor.

### BRIEF SUMMARY OF THE INVENTION

The present invention rests in part on the identification of specific cardiac proteins involved in A₃ receptor-mediated cardioprotection, termed A₃ receptor-mediated cardioprotective proteins (A₃Ps), which may play a key role in mimicking ischemic preconditioning observed with the cardioprotection. Proteins can be expressed that differ in amino acid composition, for example, as a result of alternative splicing or limited proteolysis, or as a result of differential post-translational modification such as glycosylation, phosphorylation, acylation, or both, so that proteins of identical amino acid sequence can differ in their pI, MW, or both. Such proteins are termed “isoforms”. The present invention further includes specific A₃ receptor-mediated cardioprotective protein isoforms (A₃PIs) exhibiting an altered expression in cardiac tissue in response to A₃ agonist infusion. The A₃Ps and A₃PIs of the invention are useful in a number of ways, including as targets in screening assays for identifying small molecule therapeutics capable of modulating the expression of one or more of the A₃Ps of the invention to provide cardioprotection in a subject in need thereof.

Accordingly, in a first aspect, the invention provides one or more A₃Ps or A₃PIs selected from the proteins listed in Tables 1-6. In one embodiment, the invention provides an A₃P exhibiting a decreased expression in cardiac tissue in response to infusion with an A₃ agonist, wherein the A₃P is selected from the group listed in Table 1. In another embodiment, the invention provides an A₃P exhibiting an increased expression in response to infusion with an A₃ agonist, wherein the A₃P is selected from the group listed in Table 2. In yet another embodiment, the invention provides an A₃P exhibiting an altered mobility on 2 dimensional gel electrophoresis in response to infusion with an A₃ agonist, wherein the A₃P is selected from the group listed in Table 3.

In another embodiment, the invention provides an A₃PI exhibiting a decreased expression in cardiac tissue in response to infusion with an A₃ agonist, where the A₃PI is selected from the group listed in Table 4 (SEQ ID NO: 1-8). In another embodiment, the invention provides an A₃PI exhibiting an increased expression in response to infusion with an A₃ agonist, wherein the A₃PI is selected from the group listed in Table 5 (SEQ ID NOs:942). In yet another embodiment, the invention provides an A₃PI exhibiting an altered mobility on 2 dimensional gel electrophoresis in response to infusion with an A₃ agonist, wherein the A₃PI is selected from the group listed in Table 6 (SEQ ID NOs:43-70).

The A₃Ps and A₃PIs of the instant invention are also therapeutically useful in providing cardioprotection to a subject in need thereof. Accordingly, in a second aspect, the invention provides pharmaceutical compositions comprising one or more of the A₃ receptor-mediated cardioprotective proteins listed in Tables 1-6, and a pharmaceutically acceptable carrier, vehicle, or diluent.

In a third related aspect, the invention provides a method of providing A₃ receptor-mediated cardioprotection in a subject in need thereof, comprising administering a therapeutically effective amount of one or more of the A₃ receptor-mediated cardioprotective proteins listed in Tables 1-6.

The A₃Ps and A₃PIs of the invention are useful separately and in any suitable combination, as targets in screening assays for identifying agents, e.g., small molecules, capable of modulating the expression of one or more A₃P and A₃PI. Such identified agents are useful therapeutics providing cardioprotection in a subject in need thereof. Accordingly, in a fourth aspect, the invention provides a method of identifying an agent capable of modulating the expression of an A₃P or A₃PI, comprising (a) contacting a first population of cells expressing an A₃P or A₃PI with a candidate agent, (b) contacting a second population of cells expressing the A₃P or A₃PI with a control agent, and (c) comparing the level of the A₃P or A₃PI in the first and second populations of cells. In one embodiment, the level of the A₃P or A₃PI is greater in the first population of cells than in the second population of cells. In another embodiment, the level of the A₃P or A₃PI is less in the first population of cells than in the second population of cells. In a more specific embodiment, the level of the A₃P or A₃PI is determined by measurement of the corresponding mRNA.

In a fifth aspect, the invention provides a method of screening for or identifying agents that modulate the expression of an A₃P or A₃PI, comprising (a) administering a candidate agent to a first mammal or group of mammals; (b) administering a control agent to a second mammal or group of mammals; and (c) comparing the level of expression of an A₃P or A₃PI in the first and second groups. In one embodiment, the mammals are animal models for cardiac response and related conditions. In another embodiment, the level of expression of the A₃P or A₃PI is greater in the first group than in the second group. In another embodiment, the level of expression of the A₃P or A₃PI is less in the first group than in the second group. In yet another embodiment, the levels of the A₃P or A₃PI in the first and second groups are further compared to the level of the A₃P or A₃PI in normal control mammals. In a more specific embodiment, administration of the candidate agent modulates the level of the A₃P or A₃PI in the first group towards the levels of the A₃P or A₃PI in the second group. In a further embodiment, the mammals are human subjects having cardiac response or related clinical or coronary event(s).

In a sixth aspect, the invention provides a method of screening for or identifying agents that modulate the activity of an A₃P or A₃PI, comprising (a) in a first aliquot, contacting a candidate agent with the A₃P or A₃PI, and (b) comparing the level of the A₃P or A₃PI in the first aliquot after addition of the candidate agent with the level of the A₃P or A₃PI in a control aliquot, or with a previously determined reference range. In a more specific embodiment, the A₃P or A₃PI is a recombinant protein.

The A₃Ps and A₃PIs of the instant invention are also diagnostically useful to assess cardioprotective capacity in a subject in need thereof. Assays detecting these proteins may be used to assess disease severity, disease progression, and response to therapy. Such assays will also augment existing diagnostic methodologies and allow identification and monitoring of patients. Accordingly, in a seventh aspect, the invention provides a method for assessing cardioprotective capacity in a subject, comprising detecting the level of one or more A₃Ps and A₃PIs listed in Tables 1-6.

The A₃Ps and A₃PIs of the instant invention are also diagnostically useful as biomarkers to assess and/or monitor cardioprotective status in a subject. Accordingly, in an eighth aspect, the invention features a method for monitoring cardioprotective status in a subject, comprising detecting the level of one or more A₃Ps and A₃PIs listed in Tables 1-6.

In a ninth aspect, the invention provides antibodies specific for an A₃P or A₃PI of the invention, including polyclonal, monoclonal, humanized, chimeric, synthetic/recombinant, and bispecific antibodies capable of immunospecific binding to an A₃P or A₃PI of the invention. The antibodies of the invention are useful in a variety of ways, including in diagnostic assays for identifying the level of an A₃P or A₃PI in a biological sample, and as potential therapeutics.

In a tenth aspect, the invention provides kits that may be used in the above-recited methods, and that may comprise single or multiple preparations, or antibodies, together with other reagents, labels, substrates, if needed, and directions for use. The kit of the invention may also comprising a nucleic acid probe capable of hybridizing to RNA encoding an A₃P or A₃PI. The kids maybe used, for example, to identify the presence and/or level of an A₃P or A₃PI in a biological sample, or may be used in assays for the identification of new diagnostic and/or therapeutic agents.

Other objects and advantages will become apparent from a review of the ensuing detailed description.

### DETAILED DESCRIPTION OF THE INVENTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “A₃ receptor-mediated cardioprotective protein” includes one or more of such proteins, reference to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art. All publications and patents mentioned are incorporated herein by reference in their entireties.

Definitions

A “A₃ receptor-mediated cardioprotective protein” (“A₃P”) is a protein expressed in heart muscle which exhibits an altered expression or activity in response to infusion with an A₃ receptor agonist. The altered expression includes an increased or decreased expression relative to that observed in the absence of infusion with an A₃ receptor agonist, and altered activity includes an increased or decreased activity relative to that observed in the absence of infusion with an A₃ receptor agonist.

The A₃Ps and A₃PIs of the invention correspond to a spot detected in a 2D gel that is differentially present in a sample (e.g. a sample of cardiac tissue) from a subject treated with an A₃ agonist compared with a sample (e.g., a sample of cardiac tissue) from a subject free from exposure to an A₃ agonist. An A₃P or A₃PI is characterized by its isoelectric point (pI) and molecular weight (MW) as determined by 2D gel electrophoresis, particularly utilizing the Preferred Technology described herein. A spot, corresponding to an A₃P or A₃PI, is identified as “differentially present” in a first sample with respect to a second sample when a method for detecting the spot (e.g., 2D electrophoresis) gives a different signal when applied to the first and second samples (i.e. the signal, or apparent amount of the feature present is different in the first versus second samples).

An A₃P, (or protein isoform “A₃PI” as defined below) is “increased” in the first sample with respect to the second if the method of detection indicates that the A₃P or A₃PI is more abundant in the first sample than in the second sample, or if the A₃P or A₃PI is detectable in the first sample and substantially undetectable in the second sample, or if the A₃P or A₃PI is more frequently detectable in the first sample than in the second sample. Conversely, an A₃P or A₃PI is “decreased” in the first sample with respect to the second if the method of detection indicates that the A₃P or A₃PI is less abundant in the first sample than in the second sample, or if the A₃P or A₃PI is undetectable in the first sample and detectable in the second sample, or if the A₃P or A₃PI is detected less frequently in the first sample than in the second sample. Samples may be compared from a single patient or a group of patients, wherein a patient or patients having a particular clinical condition or a characteristic symptom or history associated with a clinical condition are compared singly, in groups or in overlapping super groups to a patient or patients relatively free from or not likely to have such conditions. An A₃P or A₃PI may also be differentially present in a sample (e.g. a sample of tissue) from a subject having cardiac response, and possibly at risk of a related clinical or coronary event, compared with a sample (e.g., a sample of body tissue) from a subject not having or not likely to have a cardiac response.

The relative abundance of a spot in two samples is determined in reference to its normalized signal, in two steps as follows: first, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature or spot whose abundance is substantially invariant, within the limits of variability of the Preferred Technology, in the population of subjects being examined, e.g. the ERFs disclosed below, or (c) more preferably to the total signal detected as the sum of each of all proteins in the sample.

Secondly, the normalized signal for the spot in one sample or sample set is compared with the normalized signal for the same spot in another sample or sample set to identify spots that are “differentially present” in the first sample (or sample set) with respect to the second sample. “Fold change” includes “fold increase” and “fold decrease” and refers to the relative increase or decrease in abundance of an A₃P or A₃PI in a first sample (or sample set) compared to a second sample (or sample set). An A₃P or A₃PI fold change may be measured by any technique known to those of skill in the art, where the observed increase or decrease will vary depending upon the technique used. The skilled artisan will understand, including based on the present description, how to select, apply and interpret any such technique(s). Preferably, fold change is determined herein as described in the Examples herein.

“Isoform” refers to a polypeptide and is characterized by one or more peptide sequences of which it is comprised, and by further reference to a pI and MW as determined by 2D gel electrophoresis, particularly utilizing the Preferred Technology as described herein. A spot may be characterized as or by an isoform having a particular peptide sequence associated with its pI and MW. As depicted herein, a spot may comprise one or more protein isoforms, which have indistinguishable pI and MWs using the Preferred Technology, but which comprise distinct peptide sequences. The peptide sequence(s) of the protein isoform can be utilized to search database(s) for proteins or polypeptide fragments comprising such peptide sequence(s), for which protein it can be ascertained whether, for example, an antibody exists which may recognize the protein and/or a member of its protein family, and as such, may be used in the methods and compositions of the present invention.

The term “modulate” when used herein in reference to expression or activity of an A₃P or an A₃PI, refers to the upregulation or downregulation of the expression or activity of the A₃P or an A₃P I. Based on the present disclosure, such modulation can be determined by assays known to those of skill in the art and/or described herein.

“Diagnosis” refers to diagnosis, prognosis, monitoring, characterizing, selecting patients, including participants in clinical trials, and identifying patients at risk for or having a particular disorder or clinical event, or having experienced a disorder or clinical event, or those most likely to respond to a particular therapeutic treatment, or for assessing or monitoring a patient's response to a particular therapeutic treatment.

“Treatment” refers to therapy, prevention, amelioration, and/or prophylaxis, and particularly refers to the administration of medicine or the performance of medical procedures with respect to a patient, for either prophylaxis, or to cure or reduce the extent of (ameliorate) or likelihood of occurrence the infirmity or malady or condition or event in the instance where the patient is afflicted. For example, the A₃Ps and/or A₃PIs of the invention are useful therapeutically as target compounds for identification of molecules able to modulate their expression or activity in order to prevent or delay the onset or development of cardiac response, to prevent or delay the progression of cardiac response, or to ameliorate the symptoms of cardiac response. A therapeutically effective amount of an agent, for example, an A₃P, A₃PI, or combination(s) thereof, is an amount sufficient to achieve the desired prophylactic, ameliorative, protective, or preventive result desired.

“Agent” refers to all materials that may be used to prepare pharmaceutical and diagnostic compositions, and may include compounds, nucleic acids, polypeptides, fragments, isoforms, or other suitable materials that may be used independently or collectively for such purposes, all in accordance with the present invention.

A “cardiac response” includes and encompasses undesirable clinical conditions and events associated with heart condition or function, as well as diseases of the heart, including angina, myocardial infarction (MI), organ failure, and other forms of coronary heart disease or myocardial ischemic injury.

### GENERAL DESCRIPTION

Preferred Technology

The A₃-receptor cardioprotective proteins (A₃Ps and A₃PIs) of the invention were identified by two-dimensional electrophoresis methodology of the Preferred Technology. As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in PCT Publication No. WO 98/23950 and in corresponding U.S. Pat. No. 6,064,754, which publications are herein specifically incorporated by reference, with particular reference to the experimental protocol described therein. It should be recognized by the skilled artisan, however, that many recognized and known two-dimensional electrophoresis systems may be utilized in practicing the present invention. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882, corresponding to U.S. Pat. No. 5,616,502, and WO 99/26186, U.S. Pat. No. 6,201,628, and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

An additional preferred scanner is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations. Statistical techniques for identifying A₃Ps and A₃PIs include uni-variate differential analysis tools, Wilcoxon rank sum test, t-test, as well as other suitable statistical methods.

In accordance with an aspect of the present invention, the A₃Ps and A₃PIs disclosed herein have been identified by infusing subjects free from cardiac response with an adenosine-3 (A₃) agonist, and then detecting and/or measuring the presence and expression of proteins, and comparing with the same tissue type samples that have been infused with vehicle free from A₃ agonist.

As those of skill in the art will readily appreciate, the MW and pI of an identified protein or protein isoform measured by the method described herein will vary to some extent depending on the precise protocol used for each step of the 2D electrophoresis and for landmark matching. As used herein, the terms “MW” and “pI” are defined, respectively, to mean the apparent molecular weight and the apparent isoelectric point of a feature or protein isoform as measured in exact accordance with the Reference Protocol. Where the Reference Protocol is followed and when samples are run in duplicate or a higher number of replicates, variation in the measured mean pI of an A₃P or A₃PI is typically less than 3% and variation in the measured mean MW of an A₃p or A₃PI is typically less than 5%. Where the skilled artisan wishes to deviate from the Reference Protocol, calibration experiments should be performed to compare the MW and pI for each A₃p or A₃PI as detected (a) by the Reference Protocol and (b) by the deviant protocol.

Three groups of A₃Ps and three groups of A₃PIs have been identified through the methods and apparatus of the Preferred Technology. The A₃Ps and A₃PIs can be described by apparent molecular weight (MW) and isoelectric point (pI) as provided in Tables 1-6.

The first group consists of A₃Ps that are decreased in cardiac tissue infused with A₃ agonist as compared with cardiac tissue infused with vehicle free from A₃ agonist. These A₃Ps can be described by apparent molecular weight (MW) and isoelectric point (pI) as shown in Table 1.

TABLE 1
A₃Ps from cardiac tissue which are decreased by the
presence of the A3 agonist when compared to vehicle:

| | | | | Mean % Volume | | |
|---|---|---|---|---|---|---|
| Table 1 | A₃P # | pI | MW (Da) | Vehicle | A3 | p-value |
| 10154972 | A3F-182 | 5.21 | 46835 | 0.8112 | 0.1633 | 0.03365 |
| 10154986 | A3F-183 | 5.33 | 45815 | 0.8214 | 0.5995 | 0.01210 |
| 10154877 | A3F-184 | 5.12 | 54362 | 0.1355 | 0.0216 | 0.00052 |
| 10154952 | A3F-185 | 5.84 | 49074 | 0.0961 | 0.0075 | 0.00354 |
| 10155015 | A3F-186 | 8.09 | 44378 | 0.0852 | NA | 0.01392 |
| 10154904 | A3F-187 | 5.05 | 52437 | 0.1118 | 0.0326 | 0.00735 |
| 10155047 | A3F-188 | 7.49 | 42706 | 0.0753 | NA | 0.02084 |
| 10154844 | A3F-189 | 7.27 | 58778 | 0.0887 | 0.0138 | 0.00918 |
| 10155388 | A3F-190 | 5.42 | 23145 | 0.0682 | NA | 0.01031 |
| 10154710 | A3F-191 | 5.16 | 85334 | 0.0658 | NA | 0.00258 |
| 10154842 | A3F-192 | 7.14 | 59231 | 0.0646 | NA | 0.00008 |
| 10155346 | A3F-193 | 5.55 | 24662 | 0.1791 | 0.1168 | 0.02829 |
| 10155110 | A3F-194 | 5.49 | 40250 | 0.0681 | 0.0115 | 0.00972 |
| 10154941 | A3F-195 | 7.23 | 49707 | 0.0505 | NA | 0.00445 |
| 10154623 | A3F-196 | 6.41 | 166044 | 0.1175 | 0.0707 | 0.03049 |
| 10154765 | A3F-197 | 6.31 | 71317 | 0.0419 | NA | 0.01319 |
| 10154896 | A3F-198 | 5.13 | 52989 | 0.0402 | NA | 0.00401 |
| 10155003 | A3F-199 | 5.84 | 44742 | 0.0375 | NA | 0.00226 |
| 10154984 | A3F-200 | 7.45 | 46330 | 0.0375 | NA | 0.04814 |
| 10155190 | A3F-201 | 5.57 | 35113 | 0.0425 | 0.0076 | 0.00468 |
| 10155133 | A3F-202 | 7.56 | 38814 | 0.0347 | NA | 0.00161 |
| 10155129 | A3F-203 | 8.84 | 39023 | 0.0317 | NA | 0.00390 |
| 10155328 | A3F-204 | 5.83 | 25854 | 0.0712 | 0.0408 | 0.04409 |
| 10154996 | A3F-205 | 7.72 | 44989 | 0.1060 | 0.0771 | 0.03439 |
| 10155211 | A3F-206 | 5.61 | 34056 | 0.0277 | NA | 0.00044 |
| 10154817 | A3F-207 | 5.00 | 61302 | 0.0271 | NA | 0.00210 |
| 10154935 | A3F-208 | 6.98 | 50109 | 0.0268 | NA | 0.03203 |
| 10154815 | A3F-209 | 6.74 | 61460 | 0.0268 | NA | 0.00011 |
| 10154868 | A3F-210 | 5.05 | 55828 | 0.0498 | 0.0231 | 0.01361 |
| 10154876 | A3F-211 | 5.08 | 54673 | 0.0240 | NA | 0.00933 |
| 10154783 | A3F-212 | 7.25 | 68261 | 0.0234 | NA | 0.03459 |
| 10154975 | A3F-213 | 6.43 | 46869 | 0.0190 | NA | 0.04059 |
| 10154898 | A3F-214 | 7.59 | 52701 | 0.0187 | NA | 0.03597 |
| 10155254 | A3F-215 | 7.56 | 30495 | 0.0304 | 0.0118 | 0.01452 |
| 10155300 | A3F-216 | 6.42 | 27574 | 0.0184 | NA | 0.01557 |
| 10155111 | A3F-217 | 5.41 | 40149 | 0.0235 | 0.0069 | 0.03598 |
| 10154840 | A3F-218 | 5.47 | 59337 | 0.0205 | 0.0044 | 0.00171 |
| 10154837 | A3F-219 | 5.14 | 59443 | 0.0234 | 0.0076 | 0.01376 |
| 10154759 | A3F-220 | 6.25 | 72141 | 0.0219 | 0.0064 | 0.04259 |
| 10154769 | A3F-221 | 5.60 | 70687 | 0.0150 | NA | 0.00490 |
| 10155377 | A3F-222 | 5.39 | 23634 | 0.0144 | NA | 0.00020 |
| 10154705 | A3F-223 | 5.69 | 86960 | 0.0228 | 0.0089 | 0.02718 |
| 10154704 | A3F-224 | 5.62 | 88006 | 0.0128 | NA | 0.04469 |
| 10154653 | A3F-225 | 5.20 | 116723 | 0.0116 | NA | 0.03422 |
| 10155217 | A3F-226 | 5.87 | 33739 | 0.0144 | 0.0029 | 0.02264 |
| 10155096 | A3F-227 | 6.48 | 41030 | 0.0113 | NA | 0.01909 |
| 10155095 | A3F-228 | 5.67 | 41070 | 0.0111 | NA | 0.02006 |
| 10155042 | A3F-229 | 6.24 | 43421 | 0.0104 | NA | 0.04451 |
| 10154991 | A3F-230 | 7.14 | 45382 | 0.0103 | NA | 0.04652 |
| 10154863 | A3F-231 | 4.82 | 56480 | 0.0101 | NA | 0.00117 |
| 10154802 | A3F-232 | 5.49 | 64025 | 0.0101 | NA | 0.01477 |
| 10154967 | A3F-233 | 5.12 | 47612 | 0.0093 | NA | 0.04837 |
| 10155448 | A3F-234 | 5.47 | 19685 | 0.0092 | NA | 0.00705 |
| 10155401 | A3F-235 | 5.46 | 22871 | 0.0125 | 0.0037 | 0.00415 |
| 10155295 | A3F-236 | 7.33 | 27780 | 0.0088 | NA | 0.02731 |
| 10155068 | A3F-237 | 4.88 | 41829 | 0.0072 | NA | 0.00589 |
| 10154873 | A3F-238 | 5.46 | 55127 | 0.0072 | NA | 0.01390 |
| 10154854 | A3F-239 | 5.82 | 57538 | 0.0071 | NA | 0.00121 |
| 10154711 | A3F-240 | 5.93 | 86011 | 0.0103 | 0.0034 | 0.03830 |
| 10154780 | A3F-241 | 5.13 | 68578 | 0.0067 | NA | 0.04131 |
| 10155216 | A3F-242 | 4.78 | 33862 | 0.0067 | NA | 0.01168 |
| 10154956 | A3F-243 | 5.40 | 48739 | 0.0065 | NA | 0.04910 |
| 10155353 | A3F-244 | 5.60 | 24387 | 0.0095 | 0.0032 | 0.04065 |
| 10155298 | A3F-245 | 6.31 | 27618 | 0.0121 | 0.0059 | 0.04085 |
| 10154872 | A3F-246 | 5.42 | 55101 | 0.0061 | NA | 0.02114 |
| 10155468 | A3F-247 | 5.18 | 17136 | 0.0061 | NA | 0.01234 |
| 10155172 | A3F-248 | 7.98 | 35720 | 0.0060 | NA | 0.04976 |
| 10155165 | A3F-249 | 5.52 | 36357 | 0.0058 | NA | 0.00258 |
| 10155230 | A3F-250 | 5.27 | 32908 | 0.0048 | NA | 0.00806 |
| 10154982 | A3F-251 | 6.04 | 46294 | 0.0040 | NA | 0.00014 |
| 10155375 | A3F-252 | 6.40 | 23675 | 0.0069 | 0.0030 | 0.04747 |
| 10155157 | A3F-253 | 6.44 | 37417 | 0.0038 | NA | 0.02428 |
| 10154983 | A3F-254 | 6.66 | 46412 | 0.0036 | NA | 0.04771 |
| 10154779 | A3F-255 | 5.23 | 68738 | 0.0028 | NA | 0.04544 |
| 10154882 | A3F-256 | 6.33 | 54666 | 0.0082 | 0.0054 | 0.03962 |
| 10155151 | A3F-257 | 7.59 | 37775 | 0.0055 | 0.0028 | 0.04621 |

The second group consists of A₃Ps that are increased in cardiac tissue infused with A₃ agonist as compared with cardiac tissue infused with vehicle free from A₃ agonist. These A₃Ps can be described by apparent MW pI as follows (Table 2):

TABLE 2
A₃Ps from cardiac tissue which are increased by the
presence of the A3 agonist when compared to vehicle:

| | | | | Mean % Volume | | |
|---|---|---|---|---|---|---|
| Table 2 | A₃P # | pI | MW (Da) | Vehicle | A3 | p-value |
| 10155628 | A3F-258 | 6.39 | 78493 | NA | 0.0014 | 0.03900 |
| 10155678 | A3F-259 | 6.20 | 57681 | NA | 0.0024 | 0.00389 |
| 10155697 | A3F-260 | 5.58 | 52323 | 0.0029 | 0.0063 | 0.03067 |
| 10155717 | A3F-261 | 5.27 | 47283 | NA | 0.0056 | 0.00691 |
| 10155905 | A3F-262 | 8.82 | 20402 | NA | 0.0060 | 0.04144 |
| 10155609 | A3F-263 | 5.70 | 95612 | NA | 0.0060 | 0.00418 |
| 10155625 | A3F-264 | 6.63 | 78863 | NA | 0.0061 | 0.00013 |
| 10155911 | A3F-265 | 5.32 | 19206 | NA | 0.0062 | 0.00805 |
| 10155902 | A3F-266 | 9.13 | 20958 | NA | 0.0064 | 0.00227 |
| 10155726 | A3F-267 | 5.58 | 45680 | NA | 0.0064 | 0.00002 |
| 10155326 | A3F-268 | 8.42 | 26042 | 0.0236 | 0.0305 | 0.04208 |
| 10155814 | A3F-269 | 4.77 | 32465 | NA | 0.0071 | 0.04977 |
| 10155666 | A3F-270 | 7.51 | 61357 | NA | 0.0074 | 0.02751 |
| 10155709 | A3F-271 | 5.54 | 49523 | NA | 0.0075 | 0.00003 |
| 10154875 | A3F-272 | 6.79 | 55191 | 0.0027 | 0.0106 | 0.04542 |
| 10155633 | A3F-273 | 7.81 | 75641 | NA | 0.0082 | 0.00005 |
| 10155793 | A3F-274 | 5.88 | 37740 | NA | 0.0084 | 0.03803 |
| 10155741 | A3F-275 | 6.07 | 42028 | NA | 0.0085 | 0.03377 |
| 10155924 | A3F-276 | 5.84 | 15827 | NA | 0.0089 | 0.00849 |
| 10155842 | A3F-277 | 6.46 | 27209 | NA | 0.0092 | 0.02494 |
| 10155769 | A3F-278 | 6.28 | 39386 | NA | 0.0093 | 0.00940 |
| 10154959 | A3F-279 | 5.54 | 48672 | 0.0049 | 0.0147 | 0.02489 |
| 10155837 | A3F-280 | 7.36 | 27625 | 0.0142 | 0.0243 | 0.04907 |
| 10155621 | A3F-281 | 5.69 | 81482 | NA | 0.0105 | 0.01404 |
| 10155724 | A3F-282 | 8.46 | 46014 | NA | 0.0106 | 0.00974 |
| 10155602 | A3F-283 | 5.18 | 109598 | NA | 0.0121 | 0.04101 |
| 10154965 | A3F-284 | 5.10 | 47972 | 0.0107 | 0.0252 | 0.02569 |
| 10154848 | A3F-285 | 8.82 | 58196 | 0.0318 | 0.0469 | 0.04776 |
| 10155681 | A3F-286 | 7.21 | 56645 | NA | 0.0152 | 0.00000 |
| 10155592 | A3F-287 | 5.70 | 134766 | NA | 0.0152 | 0.03676 |
| 10155622 | A3F-288 | 6.21 | 80693 | NA | 0.0154 | 0.00020 |
| 10155368 | A3F-289 | 9.01 | 23896 | 0.0608 | 0.0768 | 0.01038 |
| 10155679 | A3F-290 | 5.13 | 57273 | NA | 0.0168 | 0.00879 |
| 10155693 | A3F-291 | 5.36 | 52845 | 0.0035 | 0.0213 | 0.03577 |
| 10154847 | A3F-292 | 8.63 | 58180 | 0.0140 | 0.0337 | 0.01967 |
| 10155647 | A3F-293 | 5.62 | 67267 | NA | 0.0201 | 0.01543 |
| 10155811 | A3F-294 | 5.61 | 33650 | NA | 0.0222 | 0.00289 |
| 10155742 | A3F-295 | 6.23 | 42004 | 0.0090 | 0.0318 | 0.02382 |
| 10155213 | A3F-296 | 6.65 | 34111 | 0.0095 | 0.0326 | 0.01316 |
| 10155668 | A3F-297 | 7.05 | 60686 | NA | 0.0290 | 0.00071 |
| 10155546 | A3F-298 | 4.81 | 11789 | 0.1118 | 0.1415 | 0.03361 |
| 10155506 | A3F-299 | 4.51 | 13158 | 0.1340 | 0.1649 | 0.02101 |
| 10155702 | A3F-300 | 7.22 | 51001 | 0.0119 | 0.0440 | 0.03085 |
| 10155718 | A3F-301 | 5.84 | 46766 | NA | 0.0334 | 0.01463 |
| 10155900 | A3F-302 | 6.16 | 21365 | NA | 0.0346 | 0.00000 |
| 10155648 | A3F-303 | 6.32 | 66975 | NA | 0.0367 | 0.00749 |
| 10155872 | A3F-304 | 5.57 | 23887 | NA | 0.0396 | 0.00001 |
| 10155437 | A3F-305 | 8.84 | 21072 | 0.0713 | 0.1119 | 0.04393 |
| 10155725 | A3F-306 | 5.32 | 45768 | NA | 0.0409 | 0.00163 |
| 10155856 | A3F-307 | 6.05 | 25642 | NA | 0.0430 | 0.02440 |
| 10154818 | A3F-308 | 7.14 | 61256 | 0.0273 | 0.0721 | 0.00049 |
| 10155268 | A3F-309 | 9.07 | 29621 | 0.0406 | 0.0902 | 0.04047 |
| 10155670 | A3F-310 | 7.30 | 60016 | 0.0214 | 0.0732 | 0.00885 |
| 10155879 | A3F-311 | 5.43 | 23549 | NA | 0.0559 | 0.00149 |
| 10155772 | A3F-312 | 7.33 | 39097 | NA | 0.0629 | 0.00012 |
| 10155161 | A3F-313 | 5.46 | 36923 | 0.0054 | 0.0731 | 0.00239 |
| 10155771 | A3F-314 | 8.13 | 39432 | NA | 0.0703 | 0.01549 |
| 10155336 | A3F-315 | 9.90 | 25337 | 0.0993 | 0.1763 | 0.01763 |
| 10154686 | A3F-316 | 7.17 | 97183 | 0.1009 | 0.1867 | 0.03235 |
| 10155114 | A3F-317 | 8.47 | 40024 | 0.0798 | 0.1677 | 0.01042 |
| 10155222 | A3F-318 | 8.67 | 33246 | 0.1016 | 0.2020 | 0.02682 |
| 10155736 | A3F-319 | 7.58 | 43830 | NA | 0.1114 | 0.00001 |
| 10155043 | A3F-320 | 8.10 | 43442 | 0.0749 | 0.2847 | 0.04211 |
| 10155088 | A3F-321 | 7.87 | 41264 | 0.3552 | 0.6985 | 0.00294 |

The third group consists of A₃Ps exhibiting a mobility shift in cardiac tissue infused with A₃ agonist as compared with cardiac tissue infused with vehicle free from A₃ agonist. These A₃Ps can be described by apparent MW and pI as follows (Table 3):

TABLE 3
A₃Ps from tissue for which a mobility shift for A₃ versus
vehicle was noticed during visual inspection of the gel images:

| Table 3 | A₃P # | pI | MW (Da) |
|---|---|---|---|
| 10154673 | A3F-322 | 5.34 | 102986 |
| 10154682 | A3F-323 | 5.34 | 97689 |
| 10154737 | A3F-324 | 5.03 | 77643 |
| 10154747 | A3F-325 | 6.58 | 76040 |
| 10154893 | A3F-326 | 5.82 | 53188 |
| 10154894 | A3F-327 | 5.93 | 53190 |
| 10154901 | A3F-328 | 5.26 | 52214 |
| 10154908 | A3F-329 | 5.10 | 51964 |
| 10154961 | A3F-330 | 6.48 | 48478 |
| 10155036 | A3F-331 | 5.30 | 43443 |
| 10155041 | A3F-332 | 5.46 | 43446 |
| 10155053 | A3F-333 | 9.04 | 42543 |
| 10155089 | A3F-334 | 5.88 | 41102 |
| 10155105 | A3F-335 | 6.30 | 40407 |
| 10155117 | A3F-336 | 9.17 | 39865 |
| 10155140 | A3F-337 | 4.61 | 38105 |
| 10155143 | A3F-338 | 8.52 | 37860 |
| 10155149 | A3F-339 | 8.74 | 37727 |
| 10155168 | A3F-340 | 6.68 | 36130 |
| 10155196 | A3F-341 | 5.70 | 34748 |
| 10155202 | A3F-342 | 5.43 | 34568 |
| 10155205 | A3F-343 | 7.91 | 34374 |
| 10155219 | A3F-344 | 5.57 | 33601 |
| 10155270 | A3F-345 | 7.59 | 29700 |
| 10155339 | A3F-346 | 5.60 | 25149 |
| 10155381 | A3F-347 | 6.04 | 23433 |
| 10155449 | A3F-348 | 9.41 | 19524 |
| 10155477 | A3F-349 | 9.04 | 16221 |
| 10155549 | A3F-350 | 6.44 | 11736 |
| 10155555 | A3F-351 | 10.70 | 11258 |
| 10155644 | A3F-352 | 5.40 | 68933 |
| 10155699 | A3F-353 | 5.15 | 51701 |
| 10155722 | A3F-354 | 7.43 | 46030 |
| 10155737 | A3F-355 | 7.73 | 43775 |
| 10155798 | A3F-356 | 8.04 | 36807 |
| 10155873 | A3F-357 | 8.51 | 23785 |
| 10156089 | A3F-358 | 7.69 | 62016 |
| 10156151 | A3F-359 | 8.24 | 44072 |
| 10156181 | A3F-360 | 8.93 | 36886 |
| 10156209 | A3F-361 | 9.29 | 29852 |
| 10156251 | A3F-362 | 7.45 | 18740 |
| 10156550 | A3F-363 | 6.97 | 96667 |
| 10156565 | A3F-364 | 8.46 | 56422 |

A₃PI

In another aspect of the invention, cardiac tissue from a subject, preferably a living subject, is analyzed for quantitative detection of one or more Adenosine 3 Receptor Agonist-Associated Protein Isoforms (A₃PIs) for screening or diagnosis of cardiac response or the extent of cardioprotection, to determine the prognosis of a subject having cardiac response, to monitor the effectiveness of cardiac response therapy, or for drug development. As is well known in the art, a given protein may be expressed as variants (isoforms) that differ in their amino acid composition (e.g., as a result of alternative splicing or limited proteolysis) or as a result of differential post-translational modification (e.g., glycosylation, phosphorylation, acylation), or both, so that proteins of identical amino acid sequence can differ in their pI, MW, or both. It follows that differential presence of a protein isoform does not require differential expression of the gene encoding the protein in question. As used herein, the term “Adenosine 3 Receptor Agonist-Associated Protein Isoform” refers to a protein isoform that is or may be differentially present in cardiac tissue that has been contacted with and/or contains an amount of an A₃ agonist that differs from control or normal, and that correspondingly, may experience a differential extent or amount of cardioprotection. This difference may, in turn, impact the presence, nature or extent of the cardiac response experienced by the subject from which the cardiac tissue is taken. It is thus possible that A₃P is may be differentially present in cardiac tissue from a subject having cardiac response compared with cardiac tissue from a subject free from cardiac response.

As will be evident to one of skill in the art, based upon the present description, a given A₃PI can be described according to the data provided for that A₃PI in Table 4-6. The A₃PI is a protein comprising a peptide sequence described for that A₃PI (preferably comprising a plurality of, more preferably all of, the peptide sequences described for that A₃PI) and has a pI of about the value stated for that A₃PI (preferably within 10%, more preferably within 5% still more preferably within 1% of the stated value) and has a MW of about the value stated for that A₃PI (preferably within 10%, more preferably within 5%, still more preferably within 1% of the stated value).

Three groups of A₃PIs have been identified by partial amino acid sequencing of A₃Fs, using the methods and apparatus of the Preferred Technology. The first group consists of A₃PIs that are decreased in the cardiac tissue infused with A₃ agonist as compared with the cardiac tissue infused with vehicle free from A₃ agonist, where the differential presence is significant. The MW, pI and partial amino acid sequences of these A₃PIs are presented in Table 4, as follows:

TABLE 4
A₃PIs from tissue which are decreased by the presence
of the A3 agonist when compared to vehicle:

| | | | | Amino Acid Sequences |
|---|---|---|---|---|
| | | | | of Tryptic Digest |
| Table 4 | A₃PI# | pI | MW (Da) | Peptides |
| 10154844 (a) | A3PI-189.1 | 7.28 | 58907 | VANEPVLAFTQGSPER |
| | | | | (SEQ ID NO: 1) |
| 10154844 (b) | A3PI-189.2 | 7.28 | 58907 | VDHTVGWPLDR (SEQ ID NO: 2) |
| | | | | ITTHYTIYPR (SEQ ID NO: 3) |
| | | | | NLSIYDGPEQR (SEQ ID NO: 4) |
| 10154952 | A3PI-185 | 5.84 | 49074 | TPAFAESVTEGDVR (SEQ ID NO: 5) |
| 10155047 | A3PI-188 | 7.49 | 42706 | VVPGYGHAVLR (SEQ ID NO: 6) |
| | | | | GLVYETSVLDPDEGIR (SEQ ID NO: 7) |
| | | | | HLPNDPMFK (SEQ ID NO: 8) |

The second group consists of A₃PIs that are increased in the cardiac tissue infused with A₃ agonist as compared with the cardiac tissue infused with vehicle free from A₃ agonist, where the differential presence is significant. The MW, pI and known homologous protein of these A₃PIs are presented in Table 5, as follows:

TABLE 5
A₃PIs from tissue which are increased by the presence
of the A3 agonist when compared to vehicle:

| | | | | Amino Acid Sequences |
|---|---|---|---|---|
| | | | | of Tryptic Digest |
| Table 5 | A3PI# | PI | MW (Da) | Peptides |
| 10155088 | A3PI-321 | 7.86 | 40906 | GTGGVDTASVGGVFDISNADR (SEQ ID |
| | | | | NO: 9) |
| | | | | FEEILTR (SEQ ID NO: 10) |
| 10155114 | A3PI-317 | 8.48 | 39795 | LNVTLPAR (SEQ ID NO: 11) |
| 10155222 | A3PI-318 | 8.68 | 32056 | IFGVTTLDIVR (SEQ ID NO: 12) |
| | | | | VDFPQDQLTALTGR (SEQ ID NO: 13) |
| | | | | GCDVVVIPAGVPR (SEQ ID NO: 14) |
| 10155268 | A3PI-309 | 9.04 | 29804 | WTEYGLTFTEK (SEQ ID NO: 15) |
| | | | | VTQSNFAVGYK (SEQ ID NO: 16) |
| | | | | SENGLEFTSSGSANTETTK |
| | | | | (SEQ ID NO: 17) |
| 10155336 | A3PI-315 | 9.69 | 24784 | QVPIDALIPGK (SEQ ID NO: 18) |
| 10155592 | A3PI-287 | 5.72 | 135248 | LIAPVAEEEATVPNNK (SEQ ID NO: 19) |
| 10155622 | A3PI-288 | 6.23 | 81613 | QLLTLSSELSQAR (SEQ ID NO: 20) |
| | | | | APDFVFYAPR (SEQ ID NO: 21) |
| 10155648 | A3PI-303 | 6.32 | 66975 | EPAAFWGPLAR (SEQ ID NO: 22) |
| | | | | SPESVALIWER (SEQ ID NO: 23) |
| 10155679 | A3PI-290 | 5.13 | 57972 | DDLYVSDAFHK (SEQ ID NO: 24) |
| | | | | LPGIVAEGR (SEQ ID NO: 25) |
| | | | | EVPLNTIIFMGR (SEQ ID NO: 26) |
| 10155718 | A3PI-302 | 5.84 | 46766 | GLVVPVIR (SEQ ID NO: 27) |
| | | | | TPAFAESVTEGDVR (SEQ ID NO: 5) |
| 10155725 | A3PI-306 | 5.32 | 45768 | SENEEFVEVGR (SEQ ID NO: 28) |
| 10155742 | A3PI-295 | 6.25 | 41766 | LEEGPPVTTVLTR (SEQ ID NO: 29) |
| | | | | LPCIFICENNR (SEQ ID NO: 30) |
| 10155772 | A3PI-312 | 7.33 | 39097 | FAFDYATK (SEQ ID NO: 31) |
| | | | | HNNLDLVIIR (SEQ ID NO: 32) |
| 10155856 | A3PI-307 | 6.06 | 25527 | ASGANFEYIIAEK (SEQ ID NO: 33) |
| | | | | NNTVGLIQLNRPK (SEQ ID NO: 34) |
| | | | | SLAMEMVLTGDR (SEQ ID NO: 35) |
| | | | | LFYSTFATDDR (SEQ ID NO: 36) |
| 10155872 | A3PI-304 | 5.57 | 23887 | LATQSNEITIPVTFESR (SEQ ID NO: 37) |
| | | | | LFDQAFGLPR (SEQ ID NO: 38) |
| | | | | GPSWDPFR (SEQ ID NO: 39) |
| 10155879 | A3PI-311 | 5.41 | 23855 | THLAPYSDELR (SEQ ID NO: 40) |
| | | | | AKPALEDLR (SEQ ID NO: 41) |
| | | | | LSPLGEEVR (SEQ ID NO: 42) |

The third group consists of A₃PIs exhibiting a mobility shift in cardiac tissue infused with A₃ agonist as compared with cardiac tissue infused with vehicle free from A₃ agonist. These A₃PIs can be described by apparent MW and pI as follows (Table 6):

TABLE 6
A₃PIs from tissue which exhibit a mobility
shift for A3 versus vehicle:

| | | | | Amino Acid Sequences |
|---|---|---|---|---|
| | | | | of Tryptic Digest |
| Table 6 | A₃PI # | pI | MW (Da) | Peptides |
| 10154673 | A3PI-322 | 5.34 | 103399 | AGTQIENIEEDFR (SEQ ID NO: 43) |
| | | | | ETADTDTAEQVIASFR (SEQ ID NO: 44) |
| 10154682 | A3PI-323 | 5.34 | 95378 | ETADTDTAEQVIASFR (SEQ ID NO: 44) |
| 10154893 (a) | A3PI-326.1 | 5.85 | 51071 | TPAFAESVTEGDVR (SEQ ID NO: 5) |
| 10154893 (b) | A3PI-326.2 | 5.85 | 51071 | MISSYVGENAEFER (SEQ ID NO: 45) |
| 10154901 | A3PI-328 | 5.25 | 52559 | QVEVLTNQR (SEQ ID NO: 46) |
| | | | | VELQELNDR (SEQ ID NO: 47) |
| | | | | VAELYEEELR (SEQ ID NO: 48) |
| | | | | FLEQQNAALAAEVNR (SEQ ID NO: 49) |
| 10154908 | A3PI-329 | 5.09 | 51284 | EIIDLVLDR (SEQ ID NO: 50) |
| | | | | AVFVDLEPTVIDEVR (SEQ ID NO: 51) |
| 10155143 | A3PI-338 | 8.53 | 39259 | PYQYPALTPEQK (SEQ ID NO: 52) |
| | | | | LQSIGTENTEENR (SEQ ID NO: 53) |
| 10155149 | A3PI-339 | 8.72 | 38264 | ELSDIAHR (SEQ ID NO: 54) |
| | | | | PYQYPALTPEQK (SEQ ID NO: 52) |
| | | | | LQSIGTENTEENR (SEQ ID NO: 53) |
| | | | | FSHEEIAMATVTALR (SEQ ID NO: 55) |
| 10155196 | A3PI-341 | 5.7 | 34686 | IVVVTAGVR (SEQ ID NO: 56) |
| | | | | LKDDEVAQLK (SEQ ID NO: 57) |
| | | | | SADTLWDIQK (SEQ ID NO: 58) |
| | | | | LIAPVAEEEATVPNNK (SEQ ID NO: 19) |
| 10155205 | A3PI-343 | 7.91 | 34374 | QDIAFAYQR (SEQ ID NO: 59) |
| | | | | AYTNFDAER (SEQ ID NO: 60) |
| | | | | TNQELQEINR (SEQ ID NO: 61) |
| | | | | AEDGSVIDYELIDQDAR |
| | | | | (SEQ ID NO: 62) |
| 10155270 | A3PI-345 | 7.6 | 29333 | VNNSSLIGVGYTQTLR (SEQ ID NO: 63) |
| | | | | LTFDTTFSPNTGK (SEQ ID NO: 64) |
| | | | | YQLDPTASISAK (SEQ ID NO: 65) |
| | | | | NNFAVGYR (SEQ ID NO: 66) |
| 10155449 | A3PI-348 | 9.26 | 19478 | YENEVALR (SEQ ID NO: 67) |
| 10155699 | A3PI-353 | 5.15 | 50302 | VELQELNDR (SEQ ID NO: 47) |
| | | | | VAELYEEELR (SEQ ID NO: 48) |
| | | | | FLEQQNAALAAEVNR (SEQ ID NO: 49) |
| 10155737 (a) | A3PI-355.1 | 7.73 | 43775 | VVIGMDVAASEFYR (SEQ ID NO: 68) |
| 10155737 (b) | A3PI-355.2 | 7.73 | 43775 | GLAPEQPVTLR (SEQ ID NO: 69) |
| | | | | GETLPPVGVNR (SEQ ID NO: 70) |

Table 7 provides human and non-human accession numbers for A₃PIs:

TABLE 7
Corresponding Accession Numbers for A₃PI

| | | | Accession |
|---|---|---|---|
| | | | number |
| | | | for Human |
| Table 7 | A3PI# | Accession number | Sequence |
| 10154844 (a) | A3PI-189.1 | — | P30038 |
| 10154844 (b) | A3PI-189.2 | P55931 (pig) | Q16134 |
| 10154952 | A3PI-185 | 7939586 (pig) | P36957 |
| 10155047 | A3PI-188 | — | O75390 |
| 10155088 | A3PI-321 | O77814 (rabbit) | P06732 |
| 10155114 | A3PI-317 | 7442964 (Mycobacterium | — |
| | | tuberculosis) | |
| 10155222 | A3PI-318 | P08249(mouse) | P40926 |
| 10155268 | A3PI-309 | 8745552 (pig), Q60932 | P21796 |
| | | (mouse), Q9TT15 (rabbit), | |
| | | P45879 (bovine), Q9Z2L0 | |
| | | (rat) | |
| 10155336 | A3PI-315 | 7297296 (Drosophila | |
| | | melanogaster) | |
| 10155592 | A3PI-287 | — | P07195 |
| 10155622 | A3PI-288 | — | 7328175 |
| 10155648 | A3PI-303 | — | 7288046 |
| 10155679 | A3PI-290 | — | P01008 |
| 10155718 | A3PI-302 | 7939586 (pig) | P36957 |
| 10155725 | A3PI-306 | P00514 (bovine), P07802 | P10644 |
| | | (pig), 8546862 (mouse), | |
| | | P09456 (rat) | |
| 10155742 | A3PI-295 | P26284 (rat), P35486 | P08559 |
| | | (mouse) | |
| 10155772 | A3PI-312 | Q28479 ( Macaca | O43837 |
| | | fascicularis (Crab eating | |
| | | macaque) (Cynomolgus | |
| | | monkey) ) | |
| 10155856 | A3PI-307 | — | P30084 |
| 10155872 | A3PI-304 | — | P04792 |
| 10155879 | A3PI-311 | P15568 ( Macaca | X00566 |
| | | fascicularis (Crab eating | |
| | | macaque) (Cynomolgus | |
| | | monkey) ) | |
| 10154673 | A3PI-322 | — | P35609 |
| 10154682 | A3PI-323 | — | P35609 |
| 10154893 (a) | A3PI-326.1 | 7939586 (pig) | P36957 |
| 10154893 (b) | A3PI-326.2 | Q29551 (pig) | — |
| 10154901 | A3PI-328 | P02540 (pig), O62654 | P17661 |
| | | (bovine) | |
| 10154908 | A3PI-329 | P02550 (pig), P02551 | — |
| | | (mouse), P05209 (chinese | |
| | | hamster), P05213 (mouse), | |
| | | P05216 (mouse) | |
| 10155143 | A3PI-338 | — | P04075 |
| 10155149 | A3PI-339 | P00883 (rabbit) | P04075 |
| 10155196 | A3PI-341 | — | P07195 |
| 10155205 | A3PI-343 | P04272 (bovine) | P07355 |
| 10155270 | A3PI-345 | Q9TT14 (rabbit), 8810222 | P45880 |
| | | (bovine), 8745554 (pig) | |
| 10155449 | A3PI-348 | — | P13645 |
| 10155699 | A3PI-353 | P02540 (pig), O62654 | P17661 |
| | | (bovine) | |
| 10155737 (a) | A3PI-355.1 | P15429 (rat), P21550 | P13929 |
| | | (mouse) | |
| 10155737 (b) | A3PI-355.2 | — | 9800545 |

Screening Assays

The present invention provides methods for identifying agents (e.g., candidate compounds or test compounds) that are capable of modulating the expression of one or more A₃Ps or A₃PIs in cardiac tissue. Agents identified through the screening method of the invention are potential therapeutics for use in providing cardioprotection to a subject in need thereof.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is herein specifically incorporated by reference in its entirety).

Examples of suitable methods based on the present description for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is herein incorporated by reference in its entirety.

Libraries of compounds may be presented, for example, presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404406; Cwirla et al. (1990) Proc. Natl. Acad. Sci. USA 87:6378-6382; and Felici (1991) J. Mol. Biol. 222:301-310), each of which is herein specifically incorporated by reference in its entirety.

In one embodiment, agents that modulate the expression of one or more A₃Ps or A₃PIs are identified in a cell-based assay system. In accordance with this embodiment, cells expressing one or more A₃Ps or A₃PIs are contacted with a candidate compound or a control compound and the ability of the candidate compound to alter expression of the A₃Ps or A3PI is determined. This assay may also be used to screen a plurality (e.g. a library) of candidate compounds. The cell, for example, can be of prokaryotic origin (e.g., E. coli) or eukaryotic origin (e.g., yeast or mammalian). Further, the cells can express the A₃Ps or A₃PI endogenously or be genetically engineered to express the A₃Ps or A₃PI. The ability of the candidate compound to alter expression of one or more A₃Ps or A₃PIs can be determined by methods known to those of skill in the art, for example, by flow cytometry, a scintillation assay, immunoprecipitation or western blot analysis.

In another embodiment, agents that modulate the expression of one or more A₃Ps or A₃PIs are identified in a cell-free assay system. In accordance with this embodiment, a native or recombinant A₃P or A₃PI is contacted with a candidate compound or a control compound and the ability of the candidate compound to modulate one or more A₃Ps or A₃PIs is determined. If desired, this assay may be used to screen a plurality (e.g. a library) of candidate compounds.

In another embodiment, a cell-based assay system is used to identify agents capable of modulating the activity of an A₃P or A₃PI protein when the A₃P or A₃PI is an enzyme. In another embodiment, a cell-free assay system is used to identify agents such as an enzyme, or a biologically active portion thereof, which is responsible for the production or degradation of an A₃P or A₃PI, or is responsible for the post-translational modification of an A₃P or A₃PI. In a primary screen, a plurality (e.g., a library) of compounds are contacted with cells that naturally or recombinantly express: (i) an A₃P or A₃PI, and (ii) a protein that is responsible for processing of the A₃P or A₃PI, in order to identify compounds that modulate the production, degradation, or post-translational modification of the A₃P or A₃PI. If desired, compounds identified in the primary screen can then be assayed in a secondary screen against cells naturally or recombinantly expressing the specific A₃P or A₃PI of interest. The ability of the candidate compound to modulate the production, degradation or post-translational modification of an A₃P or A₃PI can be determined by methods known to those of skill in the art, including without limitation, flow cytometry, a scintillation assay, immunoprecipitation and western blot analysis.

In a specific embodiment, agents that modulate (i.e., upregulate or downregulate) the expression of an A₃P or A₃PI are identified by contacting cells (e.g., cells of prokaryotic origin or eukaryotic origin) expressing the A₃P or A₃PI with a candidate compound or a control compound (e.g., phosphate buffered saline (PBS)) and determining the expression of the mRNA encoding the A₃P or A₃PI. The level of expression of a selected mRNA encoding the A₃P or A₃PI in the presence of the candidate compound is compared to the level of expression of the mRNA encoding the A₃P or A₃PI in the absence of the candidate compound (e.g., in the presence of a control compound). The candidate compound can then be identified as a modulator of the expression of the A₃P or A₃PI based on this comparison. For example, when expression of the A₃P or A3PI is significantly greater in the presence of the candidate compound than in its absence, the candidate compound is identified as a stimulator of expression of the A₃P or A₃PI. Alternatively, when expression of the A₃P or A₃PI is significantly less in the presence of the candidate compound than in its absence, the candidate compound is identified as an inhibitor of the expression of the A₃P or A₃PI. The level of expression of an A₃P or A₃PI or the encoding mRNA can be determined by methods known to those of skill in the art. For example, mRNA expression can be assessed by Northern blot analysis or RT-PCR, and protein levels can be assessed by western blot analysis.

In another embodiment, agents that modulate (i.e., upregulate or downregulate) the expression of an A₃P or A₃PI are identified in an animal model. Examples of suitable animals include, but are not limited to, mice, rats, rabbits, monkeys, guinea pigs, dogs and cats. Preferably, the animal used represents a model of cardiac response. In accordance with this embodiment, the test compound or a control compound is administered (e.g., orally, rectally or parenterally such as intraperitoneally or intravenously) to a suitable animal and the effect on the expression of the A₃P or A₃PI is determined. Changes in the expression of an A₃P or A₃PI can be assessed by the methods outlined above.

Diagnostic Methods

A₃Ps can be used for detection, prognosis, diagnosis, or monitoring of cardiac response or for drug development. In one embodiment of the invention, cardiac tissue from a subject is analyzed by 2D electrophoresis for quantitative detection of one or more of the A₃Ps listed in Tables 1 and 2. An increased abundance of one or more A₃Ps in the cardiac tissue from the subject relative to cardiac tissue from a subject or subjects free from cardiac response (e.g., a control sample or a previously determined reference range) may suggest the relative lack of cardioprotection and the consequent, increased likelihood of the occurrence or the presence of cardiac response, or the converse, absence of such cardiac response. In a preferred embodiment, cardiac tissue from a subject is analyzed for quantitative detection of a plurality of A₃Ps.

Antibodies to A₃Ps or A₃PIs

The invention provides A₃Ps or A₃PIs, and antigenic fragments thereof, which may be used as an immunogen to generate antibodies which immunospecifically bind such an immunogen. Anti-A₃P or A₃PI antibodies can be produced by methods and techniques known to one of skill in the art. The anti-A₃P or A₃PI antibodies of the invention may be used in a variety of ways, including for detection of A₃Ps or A₃PIs in an immunoassay. In one embodiment, an immunoassay is performed by contacting a sample with an anti-A₃P or A₃PI antibody under conditions such that the immunospecific binding can occur if the A₃P or A₃PI is present, and detecting or measuring the amount of immunospecific binding by the antibody. In specific embodiments, an anti-A₃PI antibody preferentially binds to the specific A₃PI rather than to other isoforms of the same protein.

A₃Ps or A₃PIs can be transferred from a gel to a suitable membrane, and subsequently probed in suitable assays that include, without limitation, competitive and non-competitive assay systems using techniques such as western blots and “sandwich” immunoassays using anti-A₃P or A₃PI antibodies as described herein, e.g., antibodies raised against the A₃Ps or A₃PIs of interest as those skilled in the art will appreciate based on the present description. The immunoblots can be used to identify those anti-A₃PI antibodies displaying the selectivity required to immuno-specifically differentiate an A₃PI from other isoforms encoded by the same gene.

Any suitable immunoassay can be used to detect a A₃P or A₃PI, including, without limitation, competitive and non-competitive assay systems using techniques such as western blots, radioiimmunoassays, ELISAs (enzyme linked immunosorbent assays), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

Kits

The invention also provides diagnostic kits, comprising an anti-A₃P or A₃PI antibody. In addition, such a kit may optionally comprise one or more of the following: (1) instructions for using the anti-A₃P or A₃PI antibody for identification A₃P or A₃PI present; (2) a labeled binding partner to the antibody; (3) a solid phase (such as a reagent strip) upon which the anti-A₃P or A₃PI antibody is immobilized; and (4) a label or insert indicating regulatory approval for diagnostic, prognostic or therapeutic use or any suitable combination thereof. If no labeled binding partner to the antibody is provided, the anti-A₃P or A₃PI antibody itself can be labeled with a detectable marker, e.g., a chemiluminescent, enzymatic, fluorescent, or radioactive moiety.

The invention also provides a kit comprising a nucleic acid probe capable of hybridizing to RNA encoding an A₃P or A₃PI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding an A₃P or A₃PI, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of A₃Ps or A₃PIs or a plurality of nucleic acids each encoding an A₃P or A₃PI. A kit can optionally further comprise a predetermined amount of an isolated A₃P or A₃PI or a nucleic acid encoding an A₃P or A₃PI, e.g., for use as a standard or control.

Statistical Analysis of Tissue Samples. One subject in the vehicle group was deemed to be a non-responder based on visual inspection of the gel images and was excluded from all statistical analyses. Average percent volumes between the A₃ and vehicle groups were compared using a two-sample t-test, however any suitable statistical method may be used. For those samples for which there was no protein detected (i.e., percent volume data absent), a value between 0 and the limit of detection was imputed, the latter being defined as the minimum percent volume recorded across all gels in the tissue experiment. Composite master images (MCIs) with p-values <=0.05 were selected. Additional MCIs were selected if a mobility shift for A₃ versus vehicle was noticed during visual inspection of the gel images.

### EXAMPLES

The following example is set forth so as to provide those of ordinary skill in the art with a complete disclosure and description of how to make and use the methods and compositions of the invention, and are not intended to limit the scope of what the inventors regard as their invention, including uses thereof. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Identification of Cardiac Proteins Exhibiting Altered Expression in Response to A₃-Agonist Infusion

Animals. Cynomolgus monkeys (Macaca fascicularis) were infused intravenously with a potent selective A₃ agonist (180 μg/kg, 5 minutes+140 μg/kg/hr, n=3) or saline vehicle (n=3). Left ventricular samples were obtained at the end of the experiment.

Statistical Analysis of Tissue Samples. Average percent volumes between the A₃ and vehicle groups were compared using a two-sample t-test. For those samples for which there was no protein detected (i.e., percent volume data absent), a value between 0 and the limit of detection was imputed, the latter being defined as the minimum percent volume recorded across all gels in the tissue experiment.

Cardiac tissue. Cardiac tissue was flash frozen and pulverized in liquid nitrogen. Samples were lyophilized prior to analysis. Prior to gel loading, tissue samples were homogenized in sample buffer containing 8 M urea/thiourea, 4% CHAPS, 1% DTT, 10 mM TRIS-HCl, pH 7.5, and centrifuged at 100,000 g for 1 hour at 10° C.

2D gel analysis. Standard 3-10 PEMs were generated on 120 μg of each tissue sample. A master group was generated containing tissue samples 2D gel images. Data from cardiac tissue samples taken from agonist-treated subjects were compared to vehicle-treated subjects to assess what tissue proteins are altered by A₃ agonist exposure.

### Example 2

### Evaluating Serum Protein Profiles for Biomarker Discovery

A study was conducted to examine the use of two-dimensional electrophoresis (2-DE) to profile serum proteins for biomarkers. Serum from a control group of primates and a group treated with an adenosine receptor agonist was obtained and examined. To increase protein resolution, 7 abundant proteins were depleted from each serum sample prior to 2-DE. Image analysis software was used to compute master gels, allowing the expression of a subset of 25 proteins to be monitored. Clustering software was applied to identify protein responses to treatment throughout the study time course. Two proteins were observed to decrease in abundance in the treated cluster set (⅔ animals), yet remained either stable, or increased in the control set (⅔ animals). An additional protein was essentially unchanged in the control set, while it showed variable expression after treatment. These results, shown in APPENDIX A, demonstrate that enriched serum samples can be used with 2-DE to observe downstream physiological responses to drug treatment.

1. An A₃ receptor-mediated cardioprotective protein (A₃P) or A₃ receptor-mediated cardioprotective protein isoform (A₃PI) exhibiting an altered expression in cardiac tissue in response to exposure to an A₃ agonist, comprising a protein selected from Tables 1-6.

2. The A₃P of claim 1, wherein the A₃P exhibits decreased expression in response to exposure to an A₃ agonist and is selected from the A₃Ps listed in Table 1.

3. The A₃P of claim 1, wherein the A₃P exhibits increased expression in response to exposure to an A₃ agonist and is selected from the A₃Ps listed in Table 2.

4. The A₃P of claim 1, wherein the A₃P exhibits an altered mobility on 2 dimensional gel electrophoresis in response to exposure to an A₃ agonist and is selected from the A₃Ps listed in Table 3.

5. The A₃PI of claim 1, wherein the A₃P exhibits decreased expression in response to exposure to an A₃ agonist and is selected from the A₃Ps listed in Table 4.

6. The A₃PI of claim 5 comprising an amino acid sequence of SEQ ID NO:1-8.

7. The A₃PI of claim 1, wherein the A₃PI exhibits increased expression in response to exposure to an A₃ agonist and is selected from the A₃PIs listed in Table 5.

8. The A₃PI of claim 7 comprising an amino acid sequence of SEQ ID NO:9-42.

9. The A₃PI of claim 1, wherein the A₃PI exhibits an altered mobility on 2 dimensional gel electrophoresis in response to exposure to an A₃ agonist and is selected from the A₃Ps listed in Table 6.

10. The A₃PI of claim 9 comprising an amino acid sequence of SEQ ID NO:43-70.

11. A pharmaceutical composition comprising one or more of an A₃P of claim 2, and a pharmaceutically acceptable carrier, vehicle or diluent.

12. A pharmaceutical composition comprising one or more of an A₃P of claim 3, and a pharmaceutically acceptable carrier, vehicle or diluent.

13. A pharmaceutical composition comprising one or more of an A₃P of claim 4, and a pharmaceutically acceptable carrier, vehicle or diluent.

14. A pharmaceutical composition comprising one or more of an A₃PI of claim 5, and a pharmaceutically acceptable carrier, vehicle or diluent.

15. A pharmaceutical composition comprising one or more of an A₃PI of claim 7, and a pharmaceutically acceptable carrier, vehicle or diluent.

16. A pharmaceutical composition comprising one or more of an A₃PI of claim 9, and a pharmaceutically acceptable carrier, vehicle or diluent.

17. A screening assay for identifying an agent capable of modulating the expression of one or more A₃P and/or A₃PI, comprising:
(a) contacting a first population of cells expressing an A₃P or A₃PI with a candidate agent;
(b) contacting a second population of cells expressing the A₃P or A₃PI with a control agent; and
(c) comparing the level of the A₃P or A₃PI in the first and second populations of cells,
wherein an agent capable of modulating the expression of one or more A₃P and/or A₃PI is identified by a difference in the level of expression of the A₃P or A₃PI in the first and second populations of cells.

18. The screening method of claim 17, wherein the level of the A₃P or A₃PI is greater in the first population of cells than in the second population of cells.

19. The screening method of claim 17, wherein the level of the A₃P or A₃PI is less in the first population of cells than in the second population of cells.

20. The screening method of claim 17, wherein the level of the A₃P or A₃PI is determined by measurement of the corresponding mRNA.

21. A method of screening for or identifying an agent capable of modulating the expression of an A₃P and/or A₃PI, comprising:
(a) administering a candidate agent to a first mammal or group of mammals;
(b) administering a control agent to a second mammal or group of mammals; and
(c) comparing the level of expression of an A₃P or A₃PI in the first and second groups,
wherein an agent capable of modulating the expression of one or more A₃P and/or A₃PI is identified by a difference in the level of expression of the A₃P or A₃PI in the first and second group of animals.

22. The method of claim 21, wherein the mammals are animal models for cardiac response and related cardiac conditions.

23. The method of claim 21, wherein the level of expression of the A₃P or A₃PI is greater in the first group than in the second group.

24. The method of claim 21, wherein the level of expression of the A₃P or A₃PI is less than in the first group than in the second group.

25. The method of claim 21, wherein the levels of the A₃P or A₃PI in the first and second groups are further compared to the level of the A₃P or A₃PI in normal control mammals.

26. The method of claim 21, wherein administration of the candidate agent modulates the level of the A₃P or A₃PI in the first group towards the levels of the A₃P or A₃PI in the second group.

27. The method of claim 21, wherein the mammals are human subjects having cardiac response or related clinical or coronary event.

28. A method of screening for or identifying agents capable of modulating the activity of an A₃P or A₃PI, comprising:
(a) in a first aliquot, contacting a candidate agent with the A₃P or A₃PI; and
(b) comparing the level of the A₃P or A₃PI in the first aliquot after addition of the candidate agent with the level of the A₃P or A₃PI in a control aliquot, or with a previously determined reference range,
wherein an agent capable of modulating the activity of one or more A₃P and/or A₃PI is identified by a difference in the activity of the A₃P or A₃PI in the first and second aliquot.

29. The method of claim 28, wherein the A₃P or A₃PI is a recombinant protein.

30. A method for assessing cardioprotective capacity in a subject, comprising detecting the level of one or more A₃Ps and A₃PIs listed in Tables 1-6.


SEQUENCE LISTING (70 sequences)

SEQ ID NO: 1 - protein, 16 aa, Homo sapiens
VANEPVLAFTQGSPER

SEQ ID NO: 2 - protein, 11 aa, Homo sapiens
VDHTVGWPLDR

SEQ ID NO: 3 - protein, 10 aa, Homo sapiens
ITTHYTIYPR

SEQ ID NO: 4 - protein, 11 aa, Homo sapiens
NLSIYDGPEQR

SEQ ID NO: 5 - protein, 14 aa, Homo sapiens
TPAFAESVTEGDVR

SEQ ID NO: 6 - protein, 11 aa, Homo sapiens
VVPGYGHAVLR

SEQ ID NO: 7 - protein, 16 aa, Homo sapiens
GLVYETSVLDPDEGIR

SEQ ID NO: 8 - protein, 9 aa, Homo sapiens
HLPNDPMFK

SEQ ID NO: 9 - protein, 21 aa, Homo sapiens
GTGGVDTASVGGVFDISNADR

SEQ ID NO: 10 - protein, 7 aa, Homo sapiens
FEEILTR

SEQ ID NO: 11 - protein, 8 aa, Homo sapiens
LNVTLPAR

SEQ ID NO: 12 - protein, 11 aa, Homo sapiens
IFGVTTLDIVR

SEQ ID NO: 13 - protein, 14 aa, Homo sapiens
VDFPQDQLTALTGR

SEQ ID NO: 14 - protein, 13 aa, Homo sapiens
GCDVVVIPAGVPR

SEQ ID NO: 15 - protein, 11 aa, Homo sapiens
WTEYGLTFTEK

SEQ ID NO: 16 - protein, 11 aa, Homo sapiens
VTQSNFAVGYK

SEQ ID NO: 17 - protein, 19 aa, Homo sapiens
SENGLEFTSSGSANTETTK

SEQ ID NO: 18 - protein, 11 aa, Homo sapiens
QVPIDALIPGK

SEQ ID NO: 19 - protein, 16 aa, Homo sapiens
LIAPVAEEEATVPNNK

SEQ ID NO: 20 - protein, 13 aa, Homo sapiens
QLLTLSSELSQAR

SEQ ID NO: 21 - protein, 9 aa, Homo sapiens
APDFVFYAP

SEQ ID NO: 22 - protein, 11 aa, Homo sapiens
EPAAFWGPLAR

SEQ ID NO: 23 - protein, 11 aa, Homo sapiens
SPESVALIWER

SEQ ID NO: 24 - protein, 11 aa, Homo sapiens
DDLYVSDAFHK

SEQ ID NO: 25 - protein, 9 aa, Homo sapiens
LPGIVAEGR

SEQ ID NO: 26 - protein, 12 aa, Homo sapiens
EVPLNTIIFMGR

SEQ ID NO: 27 - protein, 8 aa, Homo sapiens
GLVVPVIR

SEQ ID NO: 28 - protein, 11 aa, Homo sapiens
SENEEFVEVGR

SEQ ID NO: 29 - protein, 13 aa, Homo sapiens
LEEGPPVTTVLTR

SEQ ID NO: 30 - protein, 11 aa, Homo sapiens
LPCIFICENNR

SEQ ID NO: 31 - protein, 8 aa, Homo sapiens
FAFDYATK

SEQ ID NO: 32 - protein, 10 aa, Homo sapiens
HNNLDLVIIR

SEQ ID NO: 33 - protein, 13 aa, Homo sapiens
ASGANFEYIIAEK

SEQ ID NO: 34 - protein, 13 aa, Homo sapiens
NNTVGLIQLNRPK

SEQ ID NO: 35 - protein, 12 aa, Homo sapiens
SLAMEMVLTGDR

SEQ ID NO: 36 - protein, 11 aa, Homo sapiens
LFYSTFATDDR

SEQ ID NO: 37 - protein, 17 aa, Homo sapiens
LATQSNEITIPVTFESR

SEQ ID NO: 38 - protein, 10 aa, Homo sapiens
LFDQAFGLPR

SEQ ID NO: 39 - protein, 8 aa, Homo sapiens
GPSWDPFR

SEQ ID NO: 40 - protein, 11 aa, Homo sapiens
THLAPYSDELR

SEQ ID NO: 41 - protein, 9 aa, Homo sapiens
AKPALEDLR

SEQ ID NO: 42 - protein, 8 aa, Homo sapiens
LSPLGEEV

SEQ ID NO: 43 - protein, 13 aa, Homo sapiens
AGTQIENIEEDFR

SEQ ID NO: 44 - protein, 16 aa, Homo sapiens
ETADTDTAEQVIASFR

SEQ ID NO: 45 - protein, 14 aa, Homo sapiens
MISSYVGENAEFER

SEQ ID NO: 46 - protein, 9 aa, Homo sapiens
QVEVLTNQR

SEQ ID NO: 47 - protein, 9 aa, Homo sapiens
VELQELNDR

SEQ ID NO: 48 - protein, 10 aa, Homo sapiens
VAELYEEELR

SEQ ID NO: 49 - protein, 15 aa, Homo sapiens
FLEQQNAALAAEVNR

SEQ ID NO: 50 - protein, 9 aa, Homo sapiens
EIIDLVLDR

SEQ ID NO: 51 - protein, 15 aa, Homo sapiens
AVFVDLEPTVIDEVR

SEQ ID NO: 52 - protein, 12 aa, Homo sapiens
PYQYPALTPEQK

SEQ ID NO: 53 - protein, 13 aa, Homo sapiens
LQSIGTENTEENR

SEQ ID NO: 54 - protein, 8 aa, Homo sapiens
ELSDIAHR

SEQ ID NO: 55 - protein, 15 aa, Homo sapiens
FSHEEIAMATVTALR

SEQ ID NO: 56 - protein, 9 aa, Homo sapiens
IVVVTAGVR

SEQ ID NO: 57 - protein, 9 aa, Homo sapiens
LKDDEVAQL

SEQ ID NO: 58 - protein, 10 aa, Homo sapiens
SADTLWDIQK

SEQ ID NO: 59 - protein, 9 aa, Homo sapiens
QDIAFAYQR

SEQ ID NO: 60 - protein, 9 aa, Homo sapiens
AYTNFDAER

SEQ ID NO: 61 - protein, 10 aa, Homo sapiens
TNQELQEINR

SEQ ID NO: 62 - protein, 17 aa, Homo sapiens
AEDGSVIDYELIDQDAR

SEQ ID NO: 63 - protein, 16 aa, Homo sapiens
VNNSSLIGVGYTQTLR

SEQ ID NO: 64 - protein, 13 aa, Homo sapiens
LTFDTTFSPNTGK

SEQ ID NO: 65 - protein, 12 aa, Homo sapiens
YQLDPTASISAK

SEQ ID NO: 66 - protein, 8 aa, Homo sapiens
NNFAVGYR

SEQ ID NO: 67 - protein, 8 aa, Homo sapiens
YENEVALR

SEQ ID NO: 68 - protein, 14 aa, Homo sapiens
VVIGMDVAASEFYR

SEQ ID NO: 69 - protein, 11 aa, Homo sapiens
GLAPEQPVTLR

SEQ ID NO: 70 - protein, 11 aa, Homo sapiens
GETLPPVGVNR